(19) United States
(12) Patent Application Publication
(10) Pub. No.: US 2018/0360621 A1
(43) Pub. Date: Dec. 20, 2018

(54) INSERTING DEVICE OF CAGE FOR DISC SPACE BETWEEN VERTEBRAE

(71) Applicant: Soojung MOON, Lewisville, TX (US)

(72) Inventor: Soojung MOON, Lewisville, TX (US)

(21) Appl. No.: 15/879,013

(22) Filed: Jan. 24, 2018

Related U.S. Application Data
(60) Provisional application No. 62/451,045, filed on Jan. 26, 2017.

(51) Int. Cl. A61F 2/46; A61F 2/44

(52) U.S. Cl. CPC A61F 2/4611; A61F 2002/30133; A61F 2/4465; A61F 2/442

(57) ABSTRACT

The present invention relates to an interspine cage inserting device, and more particularly, to an interspine cage inserting device capable of stably and easily inserting a cage inserted between spines.

Disclosed is an interspine cage inserting device. The interspine cage inserting device includes: a support unit including an internal hollow portion and accommodating an internal rod coupled with a cage inserted between spines; a first moving unit formed on one side of the support unit and pushing the cage in connection with one side of the cage; a second moving unit formed on the other side of the support unit and connected with the other side of the cage to be connected to move backward when the first moving unit moves forward; and an adjustment unit connected to the support unit so as to adjust a rotational amount of the cage by adjusting movement of the first moving unit and the second moving unit.

### CROSS REFERENCE TO RELATED APPLICATION

This application claims the benefit of U.S. provisional patent application No. 62/451,045 filed Jan. 26, 2017, the entire disclosure of which is incorporated herein by reference.

### FIELD OF THE INVENTION

The present invention relates to an interspine cage inserting device, and more particularly, to an interspine cage inserting device capable of stably and easily inserting a cage between spines.

### BACKGROUND OF THE INVENTION

An interverbal disc as a disc of cartilage sandwiched between vertebrae of a spine performs an absorption function of absorbing a load and impact of a body between respective spines except for a part of a cervical vertebra and distributing the impact like a spring. In this case, the interverbal disc serves to hold the spine not to be separated, smooth a range of spinal articulation by separating two spines so as to prevent spinal nerves from being compressed, and make a motion of each spine.

Such an interverbal disc consists of a fibrous ring and a nucleus pulposus. The fibrous ring regulates the motion of spinal fragments and an inner nucleus consists of 70 to 80% of water. The interverbal disc cushions or transmits the load and the impact applied in a vertical direction. In degenerative disc diseases, the fibrous ring becomes weaker in their motions or an ability to accept a nucleus and a water content of the fibrous ring decreases. A complex result, leads to diseases such as spinal stenosis, osteophyte formation, disc prolapse, and nerve root compression.

As one method of treating the disease accompanied by the interspine disc, there may be a method of replacing a space between two adjacent spine supports with an artificial disc or implant, a so-called cage, after removing an interspine disc of a human body, which is damaged. That is, the method is to restore the function of the spine by restoring an original distance between two adjacent spinal supports, which is an original height of the interspine disc in order to create a natural state as possible by implanting the cage.

In recent years, transforaminal lumbar interbody fusion (TLIF) has been proposed as a surgical procedure for inserting such a cage into the spines. The TLIF as one of spine body fusion techniques is a surgical method of inserting the cage in a posterior approach. When the TLIF is specifically described, the TLIF as an operation of inserting the cage by using an insertion device while removing a spinal joint in a direction in which a neuropore comes out after the spine is incised to be small along both sides of spinal muscles and the spine is minimally exposed to fix screws has an advantage in that bleeding is fewer and an operation time can be shortened.

In the TLIF, for the minimum incision and minimization of interference in the human body, in general, a tip of the cage is first operated so as to be inserted through a rear surface (a back side) of the human body and positioned between the spines and thereafter, a lateral surface of the cage is disposed on a front surface (an abdomen side of the human body) between the spines to complete insertion of the cage. That is, an impactor, which is an auxiliary device, is required to make the lateral surface of the inserted cage face the front surface between the spines and by applying a force to the lateral surface of the cage using the impactor, and as a result, the cage can be arranged by rotating the cage. However, the operation using the impactor has a disadvantage in that it is difficult to make the operation or the success or the failure of the operation depends on an operation ability of the operator.

As a method for facilitating the adjustment of the position of the cage, there have been proposed technologies such as Korean Patent Registration No. 10-1273199 (hereinafter, referred to as ‘Patent Document 1’) and Korean Patent Registration No. 10-0371308 (hereinafter, referred to as Patent Document 2) have been proposed. The cage inserting devices of the prior art are configured to implement articulated rotational motion, the so-called articulating mechanism in which the tip of the cage is inserted facing the front surface between the spines and then, the cage is pivoted.

In the cage inserting device in which the articulating mechanism is implemented, it is necessary to maintain a standing posture of the cage without rotating the cage during the insertion of the cage and to precisely adjust the posture of the cage in the process of rotating the cage to a regular location after inserting the cage and various research and development are carried out to solve such technical problems.

### REFERENCE

### Patents

1. Korean Patent 10-1194219 (2012 Oct. 18)
2. Korean Patent 10-0371308 (2003 Jan. 23)

### DESCRIPTION OF THE INVENTION

### Problems to Solve

An object of the present invention is to provide an interspine cage inserting device capable of stably maintaining a posture of a cage at the time of inserting the cage between spines and precisely adjusting a rotational posture of the cage.

The technical objects of the present invention are not limited to the aforementioned technical objects, and other technical objects, which are not mentioned above, will be apparently appreciated by a person having ordinary skill in the art from the following description.

### Solution for the Problems

In accordance with an embodiment of the present invention, an interspine cage inserting device may include: a support unit including an internal hollow portion and accommodating an internal rod coupled with a cage inserted between spines; a first moving unit formed on one side of the support unit and pushing the cage in connection with one side of the cage; a second moving unit formed on the other side of the support unit and connected with the other side of the cage to be connected to move backward when the first moving unit moves forward; and an adjustment unit connected to the support unit so as to adjust a rotational amount of the cage by adjusting movement of the first moving unit and the second moving unit.

The interspine cage inserting device in accordance with the embodiment of the present invention may further include, in which the first moving unit and the second moving unit are formed to face each other on both sides of the support unit, a power transmission unit connecting the first moving unit and the second moving unit.

In the interspine cage inserting device in accordance with the embodiment of the present invention, a curved surface supporting part supporting a curved part of the cage may be formed at the tip of the second moving unit.

In the interspine cage inserting device in accordance with the embodiment of the present invention, the adjustment unit may include a rotation handle installed in the support unit.

In the interspine cage inserting device in accordance with the embodiment of the present invention, a power transmission unit that moves the second moving unit and the first moving unit in opposite directions to each other with rotation of the adjustment unit may be installed in the support unit.

The interspine cage inserting device in accordance with the embodiment of the present invention may further include a fixation unit that fixes and releases the second moving unit to and from a first position of the support unit at the time of initially inserting the cage between spines.

In the interspine cage inserting device in accordance with the embodiment of the present invention, the fixation unit may include an operating lever pivotably installed in the second moving unit, and a first coupling part provided in the support unit and detachably coupled to the operating lever.

In the interspine cage inserting device in accordance with the embodiment of the present invention, the fixation unit may further include a second coupling part detachably coupled to the operating lever so as to fix and release the second moving unit to and from another specific position of the support unit when the cage rotates.

In the interspine cage inserting device in accordance with the embodiment of the present invention, a label indicating a pivoting state of the cage may be displayed in the support unit and the second moving unit.

The interspine cage inserting device in accordance with the embodiment of the present invention may further include: a handle part couplable to the support unit; and a handle coupling part detachably coupling the handle part to the support unit.

In the interspine cage inserting device in accordance with the embodiment of the present invention, the handle coupling part may include insertion slots formed on both surfaces of the support formed in the support unit and protrusion insertion units formed on the end of the handle part in a pair so as to form an insertion space for the support unit and inserted into the insertion slots.

In the interspine cage inserting device in accordance with the embodiment of the present invention, the handle coupling part may further include a fixation means rotatably installed in the protrusion insertion unit and fixing or releasing the support unit.

### Advantage of the Invention

According to an exemplary embodiment of the present invention, an interspine cage inserting device has an effect of stably maintaining a posture of a cage in the process of inserting the cage through a support structure and a fixation structure by a first moving unit and a second moving unit.

Further, the first moving unit and the second moving unit are connected to each other to adjust relative movement and movement amounts of the first moving unit and the second moving unit can be adjusted through adjustment by an adjustment unit to precisely adjust the posture of the cage when the cage rotates.

Further, according to an articulated rotary motion structure using a movement mechanism of the first moving unit and the second moving unit, the cage can be rotated in a larger range than the related art.

In addition, each constituent part of the cage inserting device is separable, so that the constituent parts can be easily separated and combined without an additional mechanism after a procedure.

### BRIEF DESCRIPTION OF THE DRAWINGS

FIG. 1 is a perspective view of an interspine cage inserting device according to an exemplary embodiment of the present invention;

FIG. 2 is an exploded perspective view illustrating some components of the interspine cage inserting device illustrated in FIG. 1;

FIG. 3 is a perspective view illustrating an interspine cage illustrated in FIG. 1 and an internal rod coupled therewith;

FIG. 4 is a partial perspective illustrated showing a state in which the interspine cage rotates;

FIG. 5 is a diagram illustrating an operational state of the interspine cage inserting device according to an exemplary embodiment of the present invention;

FIG. 6 is a diagram illustrating insertion mechanism of the cage during the operation of cage inserting deice;

FIGS. 7 and 8 are diagrams illustrating a moving position of a supporting bar and position of the fixation unit thereof;

FIG. 9 is a diagram illustrating installation of a handle coupling part coupled to a interspine cage inserting device;

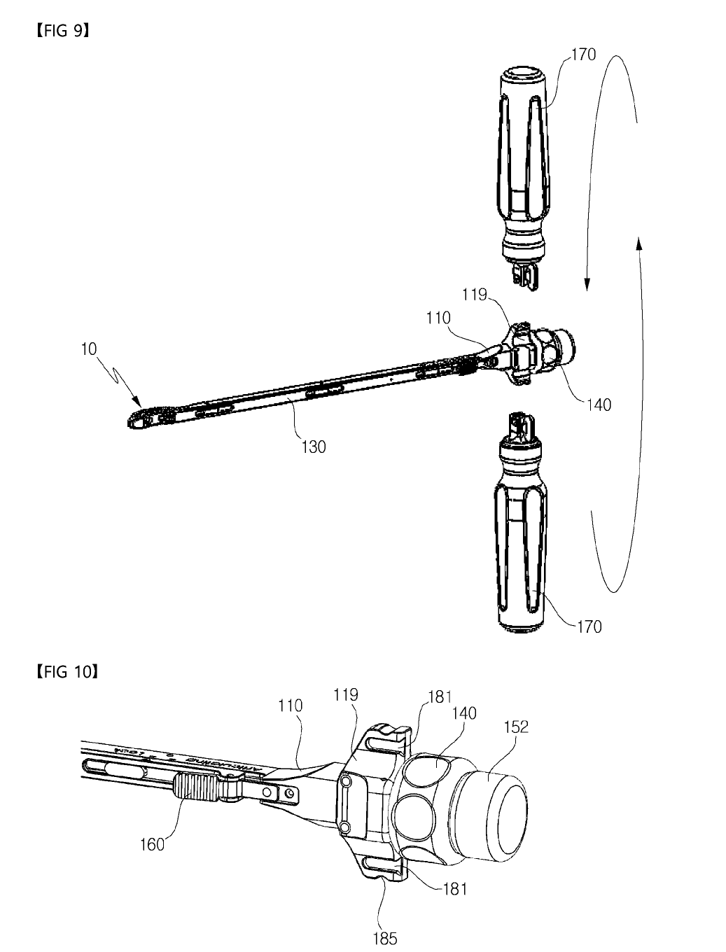

FIGS. 10 and 11 are diagrams of the handle coupling part for coupling of the handle.

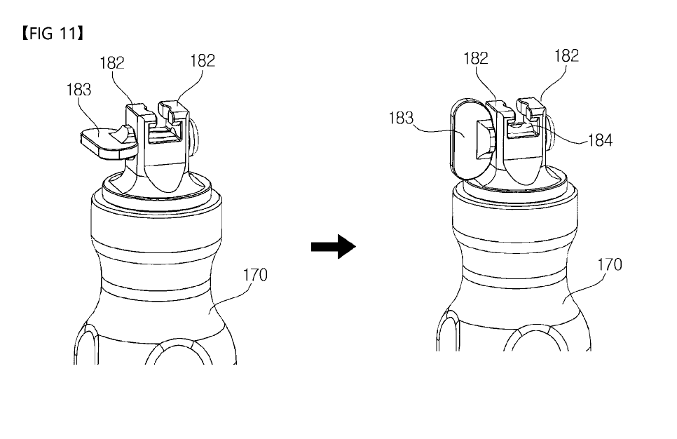

### DETAILED DESCRIPTION OF THE EMBODIMENTS

Hereinafter, an interspine cage inserting device related with the present invention will be described in more detail with reference to drawings.

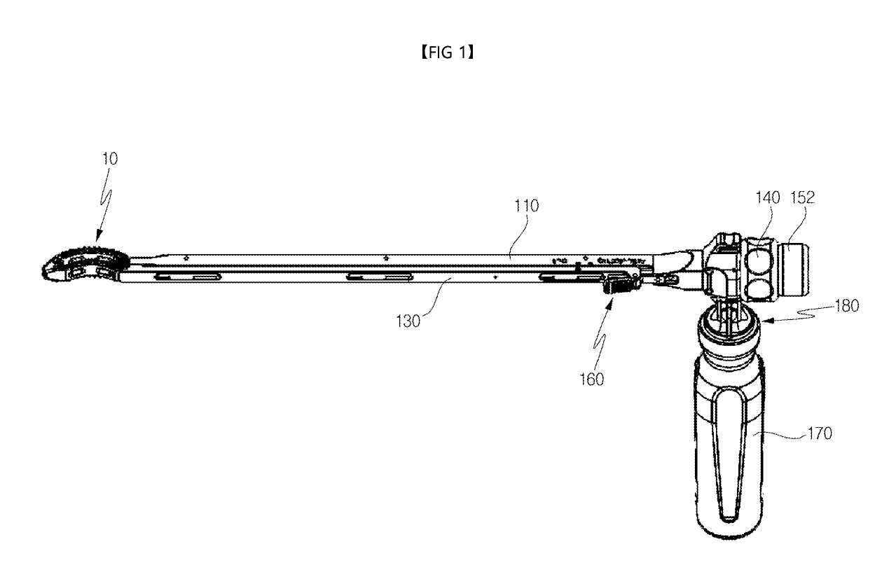

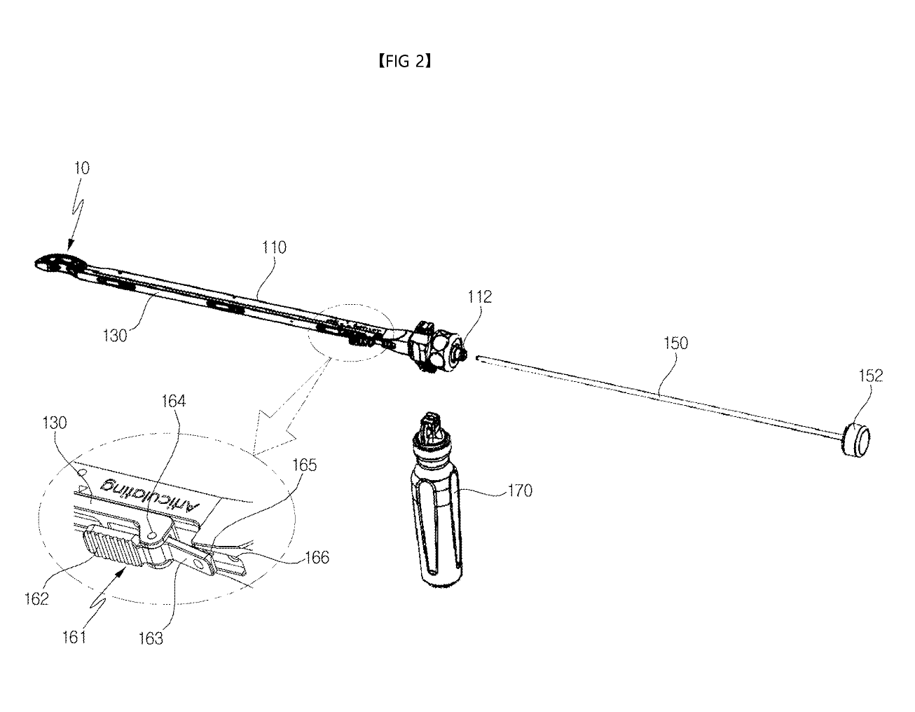

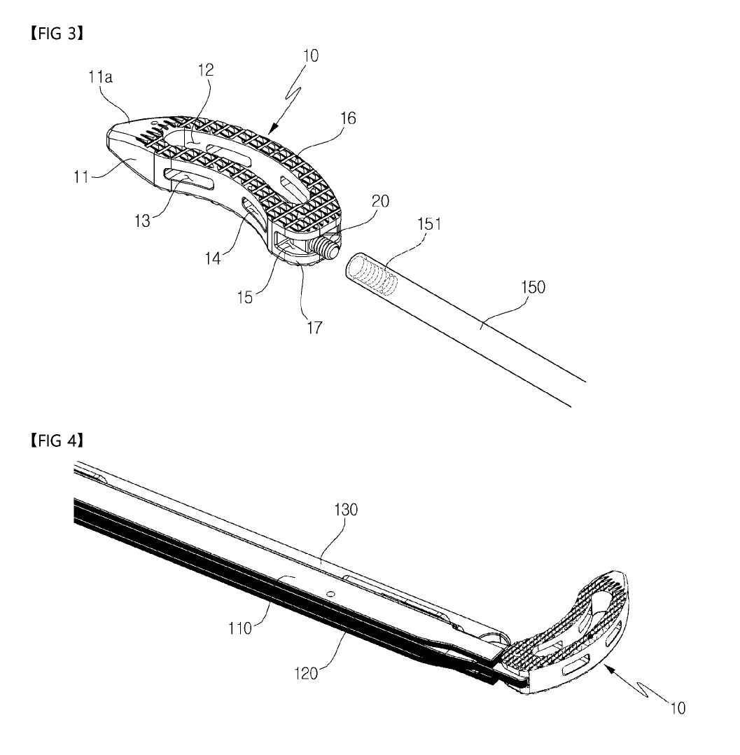

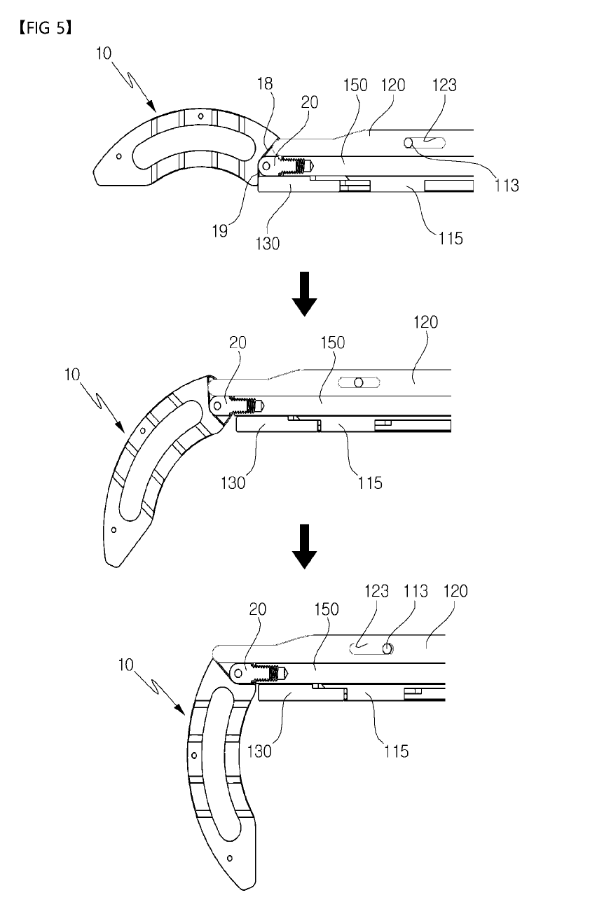

FIG. 1 is a perspective view of an interspine cage inserting device according to an exemplary embodiment of the present invention, FIG. 2 is am exploded perspective view illustrating some components of the interspine cage inserting device illustrated in FIG. 1, and FIG. 3 is a perspective view illustrating an interspine cage illustrated in FIG. 1 and an internal rod coupled therewith. FIG. 4 is a partial perspective illustrated showing a state in which the interspine cage rotates.

Referring to FIGS. 1 to 4, an interspine cage inserting device according to the present invention includes a support unit 110, a first moving unit 120, a second moving unit 130, and an adjustment unit 140.

The support unit 110 receives an internal rod 150 (see FIG. 2) that is coupled with a cage 10 to be inserted between spines into an internal hollow. Referring to FIG. 2, an opening portion 112 for communicating the inside and the outside of the support unit 10 is provided at a rear end of the support unit 10 and the internal rod 150 is inserted into the support unit 110 through the opening portion 112. A rotary knob 152 for rotating operation is provided at the rear end of the internal rod 150.

Referring to FIG. 3, the cage 10 includes a cage body 11 forming an outer appearance thereof, and a pivoting member 20 rotatably coupled thereto. The cage body 11 is provided with a slot 15 for forming a pivoting space of the pivoting member 20 at the rear end thereof. The pivoting member 20 is hinge-coupled with the cage body 11 so as to pivot inside the slot 15.

The pivoting member 20 is rotatably formed at the rear end of the cage body 11 and is connected with the internal rod 150. A male thread is formed on an outer circumferential surface of the pivoting member 20 and a female thread 151 is formed on the internal rod 150 so as to be fastened thereto. In the exemplary embodiment, the male thread is formed on the pivoting member 20 and the female thread 151 is formed on the internal rod 150. However, the present invention is not limited thereto and an opposite case is also possible and other structures may be used as long as the pivoting member 20 and the internal rod 150 are male-female fastened with each other.

Referring to FIG. 4, the first moving unit 120 is slidably installed in the support unit 110. In the case of the exemplary embodiment, a structure in which the first moving unit 120 is inserted into a slide space formed in the support unit 110 is exemplified. The first moving unit 120 is configured to push one side of the cage 10 as the first moving unit 120 moves forward from the support unit 110, that is, moves a direction toward the cage 10.

Referring back to FIGS. 1 and 2, the second moving unit 130 is configured to support the other side of the cage 10. That is, the first moving unit 120 and the second moving unit 130 are configured with both sides of the cage 10 connected with the internal rod. The second moving unit 130 is slidably connected to the supporting unit 210 in parallel to the first moving unit 120 so that the second moving unit 130 moves backward when the first moving unit 120 is moved forward so as to implement an articulated rotary of the cage 10, so-called articulating mechanism.

The adjustment unit 140 is provided in the form of a rotary handle rotatably installed at the end of the support union. In the case of exemplary embodiment, the handle-shaped unit 140 is provided at the rear end of the support unit 110. The adjustment unit 140 adjusts movement of the first moving unit 120 and the second moving unit 130.

A rotational amount of the cage 10, that is, a rotational angle of the cage support unit 110 to the pivoting member 20 may be adjusted by adjusting the movement of the first moving unit 120 and the second moving unit 130 which are slidably connected to each other according to adjustment by the adjustment unit 140. The rotary knob 152 of the internal rod 150 inserted in the hollow inside the support unit is disposed at a rear end of the adjustment unit 140. Movement amounts of the second moving unit 130 and the first moving unit 120 may be adjusted according to the rotary amount of the adjustment unit 140. When the adjustment unit 140 rotates in one direction, the second moving unit 130 may be configured to move backward and when the adjustment unit 140 rotates in an opposite direction thereto, the second moving unit 130 may be configured to move forward and the first moving unit 120 may be configured to move backward.

To this end, a power transmission unit may be installed in the support unit 10, which moves the second moving unit 130 and the first moving unit 120 in the opposite directions to each other. The power transmission unit may be configured to include a first power transmission unit for switching the linear motion of the second moving unit 130 and a second power transmitting unit for moving the linear movement of the second moving unit 130 to the first moving unit 120 and a second power transmission unit for transmitting the power to the second power transmission unit. An example of the first power transmission unit may include a combination of a worm gear and a rack-pinion, and an example of the second power transmission unit may include the rack-pinion. However, the first and second power transmission units may be modified into various forms as well as the configurations. Further, the first power transmission unit may convert a rotational motion of the adjustment unit 140 into a linear motion of the first moving unit 120 and the second power transmission unit may transmit thee linear motion of the first moving unit 120 to the second moving unit 130. In addition, the power transmission unit may be configured not to be divided into the first power transmission unit and the second power transmission unit, but to transmit the rotational motion of the adjustment unit 140 to each of the first moving unit 120 and the second moving unit 130.

Referring to FIGS. 1 and 2, the cage inserting device according to the present invention includes a fixation unit 160 that fixes and releasing the second moving unit and the support unit 110 at specific positions. The fixation unit 160 fixes the position of the second moving unit 130 supporting the cage 10 to prevent the cage 10 from rotating arbitrarily when the cage 10 is inserted, and as a result, it is possible to provide firm fixing force at the time of initial insertion.

The fixation unit 160 may have a configuration including an operating lever 161 rotatably installed in the second moving unit 130 and a first coupling unit 165 provided on the support unit 110 and the first coupling unit 165 is configured to be detachable from the operating lever 161.

The operating lever 161 is formed integrally with an operating unit 162 and the fixation unit 163 is integrally formed pivotally connected to the rear end of the second moving unit about a rotational axis 164. The fixation unit 163 may includes an insertion protrusion and in this case, the coupling unit 165 may have a shape of the insertion groove into which the insertion protrusion is inserted. The user pulls the operating unit 162 of the operating lever 161 to rotate the operating lever 161 so that the fixing unit 163 is coupled to with the first fixation unit 165 and the operating unit 162 is pushed to rotate in the opposite direction to separate the fixation unit 163 from the first coupling unit 165.

The first coupling unit 165 may be modified in various forms as long as the first coupling unit 165 is detachably coupled to the fixing unit 163 as a male-female coupling structure.

The fixation unit 160 may further include a second coupling unit 166 for fixing and releasing the second moving unit 130 to and from a second position of the support unit 110 when the cage 10 rotates. The second position corresponds to a position where the second moving unit moves backward by a predetermined distance from the first position and the second coupling unit 166 is configured to be detachably coupled to the operating lever 161 similarly to the first coupling unit 165. According to the embodiment, the second coupling unit has a shape of an insertion groove that is spaced rearward by a predetermined distance from the first coupling unit 165 which has the shape of the insertion groove. The second coupling unit serves to fix the position of the second moving unit 130 while the cage rotates at a predetermined angle to firmly maintain the state of the second moving unit 130.

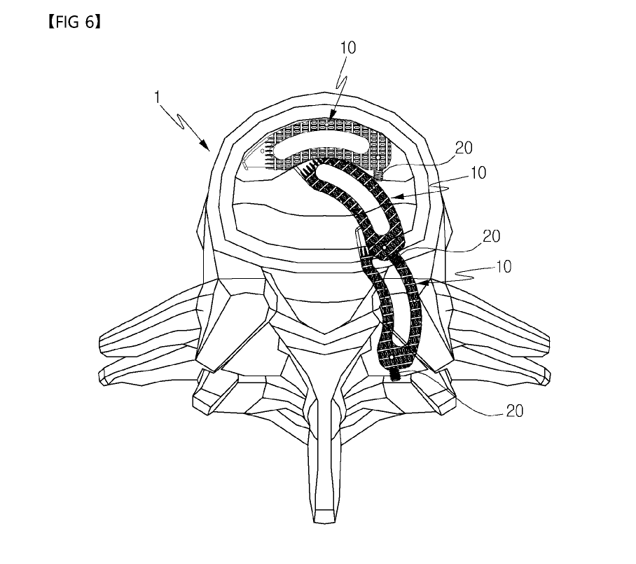

FIG. 5 is a diagram illustrating an operational state of the interspine cage inserting device according to an exemplary embodiment of the present invention.

The user rotates the rotary knob 152 of the internal rod 150 to fasten the internal rod 150 and the pivoting member 20 of the cage 10 and operates the fixation unit 160 to fix the second moving unit 130. Then, the cage inserting device enters an insertion position of a spine body 1 to insert the cage 10.

In the process of initial insertion, the first moving unit 120 supports a first support wall 18 formed in a slot 15 of the cage body 11 and the second moving unit 130 supports a second support wall 19 of the cage body 11. Since the position of the second moving unit 130 is fixed by the fixation unit 160, the cage 10 may be stably entered without being arbitrarily rotated by a strong support force when the cage 10 is initially inserted.

When fixation by the fixing unit 160 is released after the initial insertion of the cage 10, the second moving unit 130 is freely movable. When the adjustment rotates in such a state, the first moving unit 120 moves forward to push the first support wall 18 of the cage body 11 and the second moving unit 130 moves backward to provide a rotational space of the cage body 11, and as a result, an articulated rotary motion of the cage 10 is achieved. In this process, since the rotational amount of the cage 10 may be adjusted according to the rotational amount of the adjustment unit 140, the posture of the gage 10 may be precisely adjusted during surgery.

When the adjustment unit 140 continuously rotates, the cage 10 rotates to approximately 90 degrees and the second support wall 19 of the cage 10 contacts the lateral surface of the pivoting member 20 to restrict the rotation. In such a state, the cage 10 may be inserted between the spines. A guide slot 123 may be formed in the first moving unit 120 and a guide protrusion 113 that moves along the guide slot 123 may be formed in the support part 110. The guide slot 123 and the guide protrusion 113 serves to guide linear movement of the first moving unit 120 and serves as a stopper that restricts a movement range of the first moving unit 120.

The fixation unit 160 is fixed while the cage 10 rotates at approximately 90 degrees so as to fix the second moving unit 130 to the second position of the support unit 110, and as a result, the cage 10 may be inserted while maintaining a rotational state of 90 degrees. When the insertion of the cage 10 is completed, the user rotates the rotary knob 152 in the direction opposite to the rotational direction at the time of fastening, the internal rod 150 is separated from the pivoting member 120 of the cage 10 and the age inserting device is pulled out from the spine body 1 at a procedure position.

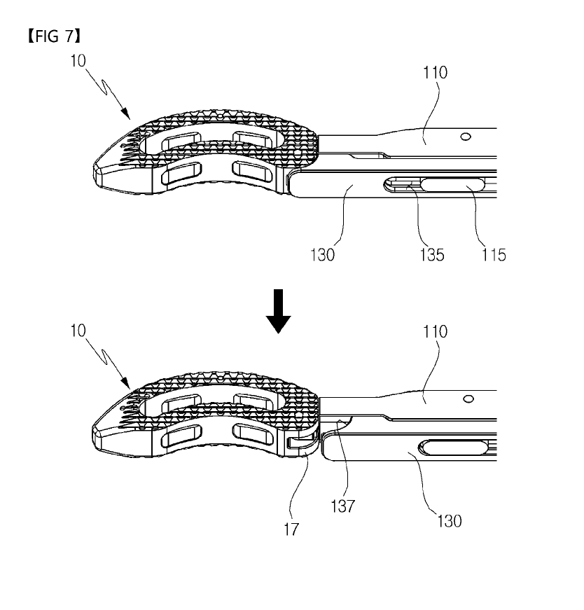

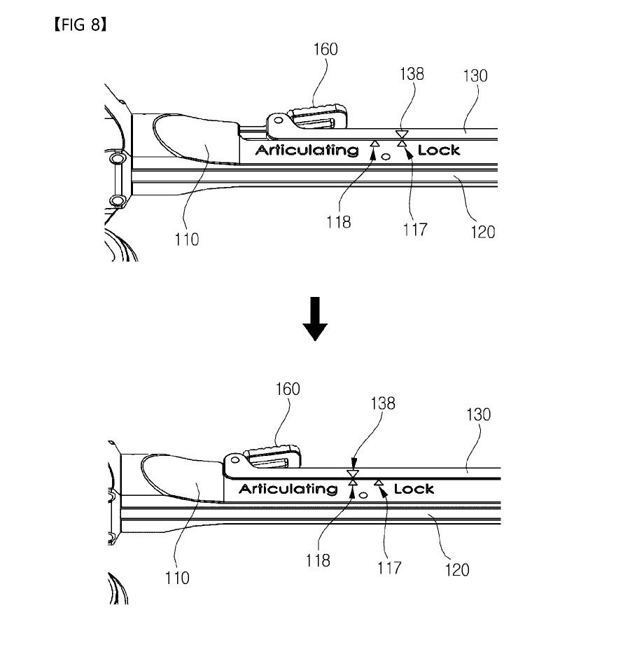

FIGS. 6 and 7 are diagrams illustrating a moving position of a second moving unit and a state of a fixation unit, respectively.

Referring to FIG. 6, a curved surface supporting part 137 for supporting a curved surface part 17 formed at the rear end of the cage 10 may be formed at the tip of the second moving unit 130 and has a shape of warping and supporting the curved surface part 17 as a shape corresponding to the curved surface part 17. With such a structure, the cage 10 may be firmly supported when the cage 10 is initially inserted.

A moving slot 115 may be formed on the lateral surface of the second moving unit 130 and a guide 115 that moves along the moving slot 135 may be provided in the support unit 110. The linear movement of the second moving unit 130 is guided and a final movement position of the second moving unit 130 is restricted by the guide structures 115 and 135.

Referring to FIG. 7, signs 117, 118, and 138 indicating the rotational state of the cage 10 may be displayed on the support unit 110 and the second moving unit 130. Therefore, the sign 138 of the second moving unit 130 indicates a label 117 corresponding to a “lock” state of the support unit 110 while the second moving unit 130 is fixed to the first position of the support unit 110 and the sign 138 of the second moving unit 130 indicates a label 118 corresponding to an “articulating” state of the support unit 110 while the second moving unit 130 is positioned at the second position of the support unit 110. According to such a configuration, it is possible to easily check the state of the cage 10, that is, whether the cage 10 is in a fixed state or in a rotated state by only checking the label without directly viewing the state of the cage 10.

Meanwhile, referring to FIGS. 1 and 2, the support unit 110 may be provided with a handle part 170 for holding by a hand and the procedure by the user. According to the embodiment, the handle part 170 may be detachably coupled to the support unit 110 and to this end, a handle coupling part 180 that detachably couples the handle part 170 and the support unit 110 may be provided between the handle part 170 and the support unit 110.

FIG. 8 is a diagram illustrating a handle coupling part for coupling of a handle part.

Referring to FIG. 8, a structure that may couple the handle part 170 may be provided in upper and lower parts of the support unit 110. Therefore, since the cage inserting device may be turned upside down depending on a position where the handle part 170 is coupled, there is an advantage that the handle part 170 may be installed and used in a direction in which both a right hand and a left hand are convenient.

Referring to FIG. 8, the handle coupling part 180 may include a pair of insertion slots 181 formed in the support unit 110 and a pair of protrusion insertion units 182 formed at the end of the handle part 170.

The support unit 110 may be provided with a support unit 119 with the insertion slot 181. The support unit 119 may be installed at the rear end of the support unit 110 and may be used as a space for accommodating the power transmission unit. The insertion slot 181 may be formed on both surfaces of the support unit 119. The support unit 119 and the insertion slot 181 are formed symmetrically on upper and lower sides of the support unit 110 so as to couple the handle part 170 to the upper and lower parts of the support unit 110.

The protrusion insertion units 182 are formed in a pair at the end of the handle part 170 to form an insertion space for the support unit 119. The protrusion insertion unit 182 is configured to be inserted into the insertion slot 181 of the support unit 119 inserted therebetween.

A fixation means 183 for fixing or releasing the support unit 119 may be rotatably installed in the protrusion insertion unit 182. According to the embodiment, the fixation means 183 has a latching part 184 which is latched on a latching groove 185 formed at a lower end of the support unit 119 and may have a configuration in which the latching part 184 rotates with the rotation of the fixation means 183 to be disposed in the latching groove 185. According to such a configuration, after the protrusion insertion unit 182 is inserted into the insertion slot 181 while the support unit 119 is inserted into a space between the pair of protrusion insertion units 182, the latching part 184 is positioned in the latching groove 185 by rotating the fixation means 183 to fix the handle part 170 to the support unit 110. Further, separation of the handle part 170 may be achieved by performing the process in a reverse order.

The interspine cage inserting device described above is not limited to the configurations and methods of the embodiments described above, but all or some of the embodiments may be selectively combined and configured so that various modifications of the interspine cage inserting device can be made.

What is claimed:

1. An interspine cage inserting device comprising:
a support unit including an internal hollow portion and accommodating an internal rod coupled with a cage inserted between spines;
a first moving unit formed on one side of the support unit and moving the cage in connection with one side of the cage;
a second moving unit formed on the other side of the support unit and connected with the other side of the cage to be connected to move backward when the first moving unit moves forward; and
an adjustment unit connected to the support unit so as to adjust a rotational amount of the cage by adjusting movement of the first moving unit and the second moving unit.

2. The interspine cage inserting device of claim 1, further comprising:
wherein the first moving unit and the second moving unit are formed to face each other on both sides of the support unit,
a power transmission unit connecting the first moving unit and the second moving unit.

3. The interspine cage inserting device of claim 1, wherein the adjustment unit includes a rotation handle installed in the support unit.

4. The interspine cage inserting device of claim 1, further comprising:
a fixation unit fixing and releasing the second moving unit to and from a specific position of the support unit at the time of inserting the case between the spines.

5. The interspine cage inserting device of claim 4, wherein the fixation unit includes
an operating lever pivotably installed in the second moving unit, and
a first coupling part provided in the support unit and detachably coupled to the operating lever.

6. The interspine cage inserting device of claim 5, wherein the fixation unit further includes a second coupling part detachably coupled to the operating lever so as to fix and release the second moving unit to and from another specific position of the support unit when the cage rotates.

7. The interspine cage inserting device of claim 1, further comprising:
a handle part couplable to the support unit; and
a handle coupling part detachably coupling the handle part to the support unit.
